(19) United States
(12) Patent Application Publication
(10) Pub. No.: US 2003/0060379 A1
(43) Pub. Date: Mar. 27, 2003

(54) Pesticides

(75) Inventors: Philip Frank Souter, Morpeth (GB); John Allen Burdis, Newcastle Upon Tyne (GB)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company, Cincinnati, OH (US)

(21) Appl. No.: 10/288,873

(22) Filed: Nov. 6, 2002

Related U.S. Application Data
(63) Continuation of application No. PCT/US01/17243, filed on May 24, 2001.

(30) Foreign Application Priority Data
May 26, 2000 (GB) 0012780.3
May 26, 2000 (GB) 0012742.3
May 26, 2000 (GB) 0012733.2

(51) Int. Cl. A61K 7/50

(52) U.S. Cl. 510/131; 510/159; 510/463

(57) ABSTRACT

An environmentally friendly soap-based pesticide having improved efficacy against aphids and other insect pests. The pesticide also has insect-repellent properties. The pesticide comprises one or more adjuncts selected from natural oils, evaporation retardants, particulate, physically-active insecticidal materials, and mixtures thereof.

### CROSS REFERENCE TO PRIOR APPLICATION

This is a continuation of International Application PCT/US01/17243, with an international filing date of May 24, 2001, and published in English.

### TECHNICAL FIELD

This invention relates to liquid pesticides and more particularly to environmentally friendly soap-based pesticides. The pesticides have improved efficacy against aphids and other insect pests. Specifically, pesticides of the invention have improved residuality, short and long-term efficacy against both immature and adult pests, and improved safety for both humans and plants and also bees and ladybirds. The invention also relates to liquid pesticidal concentrates suitable for making the liquid pesticides by dilution with water. The invention further relates to pesticides having insect-repellent properties.

### BACKGROUND OF THE INVENTION

Many different kinds of pesticides are used to kill insects. Depending on the mode of action, the insecticides can be classified in two broad groups, chemical and physical insecticides. Among chemically acting insecticides are included: i) insecticides which affect the nervous system e.g., pyrethroids, organophosphorous and carbamates; ii) insecticides which affect the endocrine or hormone system e.g., hydroprene, methoprene, pyroxyfen and fenoxycarb and iii) insecticides which inhibit exoskeleton formation e.g., benzophenyl ureas. Physically acting insecticides are believed to affect the water balance of the insects e.g., boric acid, diatomaceous earth and silica aerogels. One of the problems found when using chemically acting insecticides is that the insect can build up resistance in succeeding generations. Also they are undesirable because they can collect in food or in water resources and they can be toxic to animals and people.

The insecticidal activity of soaps is known (See U.S. Pat. No. 4,904,645 and U.S. Pat. No. 5,030,658). Soaps are naturally occurring materials having no known long-term environmental effects. Although the mode of action of the soap is not fully understood it is known that aqueous solutions of soap have a lethal effect on soft-bodies immature pests especially at high soap concentrations. Adult pests on the other hand appear to be less susceptible and longer term efficacy is relatively poor. Moreover high concentrations of soap can be detrimental to the plants themselves. U.S. Pat. No. 4,904,645 discloses an insecticidal solution for application to decorative and crop plants comprising a mixture of alkali metal salts of monocarboxylic acids, pyrethrum, a solvent for the pyrethrum and an antioxidant. U.S. Pat. No. 5,030,658 teaches the use of a sodium or potassium salt of a mixture of monocarboxylic fatty acids and a chelant agent in order to provide an arthropodicidal solution with maximum amount of active soap.

U.S. Pat. No. 4,379,168, U.S. Pat. No. 4,933,371 and U.S. Pat. No. 6,004,569 use different components of plant natural oils to kill different invertebrates. U.S. Pat. No. 4,379,168 discloses pesticide compositions containing D-limonene as insect-killing ingredient and surfactants or emulsifiers and water. The compositions are applied to rid small animals of fleas and ticks and to rid household areas of cockroaches. U.S. Pat. No. 4,933,371 describes a method of treating animal hosts and their environment to kill fleas and ticks using linalool. U.S. Pat. No. 6,004,569 discloses a pesticide and a method to kill invertebrates, the pesticide comprises a terpene having a six membered carbon ring and at least one oxygenated functional group and a carrier. There is no specific disclosure of the application of said pesticides to plants.

Inert particulate solids (with a diameter usually in the order of less than 30 μm) can also be used as insecticide. They are usually applied to plants as dried powder. However, powder-based pesticides have a number of disadvantages including: i) it is difficult to get an even distribution of particles on the target; ii) they can easily drift off target during application; iii) the residues can be easily removed from target by air movement or water; iv) they may not adhere well to the target, and v) powder can be breathed by human and animals. U.S. Pat. No. 5,693,344 discloses a powder pesticide composition and a method for delivering such composition. The pesticide is in the form of crystalline particles comprising a fragrance which kill pests by mechanical and neural methods. The crystalline particles puncture the exoskeleton, absorb body fluid of the insect and permit the entry and neural actuation of the fragrance on the insect.

One of the drawbacks of insecticidal soaps is that they seem to work better when they are wet. Therefore, it would be very valuable to find a way to keep the soap wet for as long as possible in order to increase the time during which the solution is insecticidally active.

An object of the present invention is to provide an environmentally friendly pesticide made from naturally occurring components. It is another object to provide a liquid soap-based pesticide having improved efficacy and residuality and which is easy to handle and safe to use. It is a further object to provide an environmentally friendly insecticide having insect repellent activity.

Yet another object of the present invention is to provide a liquid insecticide which is easy to handle and to apply, which acts largely by a physical mode of action, which overcomes the known problems of powder-based insecticides, which has improved residuality and efficacy, and which has an improved safety and environmental profile.

### SUMMARY OF THE INVENTION

The present invention relates in one aspect thereof to a liquid pesticide comprising soap and fragrance in levels such as to provide synergistic enhancement of insecticidal and/or other pesticidal activity. Preferred according to this aspect of the invention is a liquid pesticide in the form of a stable micellar insecticidal soap solution having the fragrance solubilised therein. In preferred embodiments, the insecticidal properties of soap and fragrance are synergistically enhanced by combining them in levels to produce an isotropic, thermodynamically stable micellar solution. The isotropic solution form of the composition also allows for a uniform delivery of the active insecticidal materials. By “thermodynamically stable” is meant a solution in which the individual soap and fragrance components cannot be separated by physical methods such as centrifugation (6000 rpm or 4000G for 15 mins at 20° C.). Of course, disperse phases other than soap and fragrance, for example thickeners, may be additionally included as described in detail hereinbelow. The outstanding performance of the present pesticide is believed to be due in part to the fact that the fragrance is in solubilised rather than emulsified form. Although the active components can be solubilised in any organic or inorganic solvent, water is preferred in the present application.

Without being bounded by the theory, it is believed that liquid pesticides of the invention are effective in killing or controlling insects and other pests by a physical rather than a biochemical mechanism such as neurotoxicity. Although the precise mechanism is not known, one possibility is that the pesticide dissolves the outer cuticle of the insect or pest leading to desiccation. Another possibility is that the pesticide forms a film over the insect or pest occluding the body surface containing the respiratory system. This film is highly wettable and evaporation occurs very slowly causing suffocation. A combination of the two mechanisms is also possible. Whatever the mechanism, it would appear that the fragrance is acting as a synergist rather than a primary insecticidal or pesticidal component. Thus none of the individual components of the fragrance are identified by the United States Environmental Protection Agency as having active insecticidal properties. Nevertheless, the fragrance is active in combination with the soap to provide a synergistic enhancement of insecticidal and pesticidal activity as demonstrated for example by the significant reduction in LC₅₀ for the combined versus the individual treatments on a range of common insects and other pests (especially foliar pests, such as aphids, whitefly, and spider mites and lawn pests such as ants, grubs, slugs and other molluscs).

LC₅₀ is defined as the lowest concentration of active (soap, fragrance or a combination thereof) required to kill 50% of insects or other pests. LC₅₀ can be calculated by plotting the number of pests killed versus concentration of active using the test method described below. In the following, the test method is described with particular reference to the treatment of pea aphids (Acyrthosiphon pisum) on bean plants (Vicia faba) but the test can be adapted to other pest species as appropriate. The test is carried out in a glasshouse at a temperature of between 16 and 25° C. at 95% relative humidity. Supplementary lighting is set to a cycle of 16 hours light and 8 hours dark provided by 400W Phillips HPI-T metal halide lamps. The test is carried out on plant pots of approximately 5 cm, each containing a young bean plant with 2 to 4 leaves in a loam based compost. A filter paper is positioned around the base of the plant to catch dead pests. 25 aphids are placed on each plant and allowed to settle overnight. The test materials and control (water) are applied to the plants up to incipient run-off using a hand held sprayer (Hozelock Polyspray II). The plants are sprayed from a distance of 20 cm, the nozzle output velocity being 150 ml/min. Counts of dead pests are made 24, 48 and 72 hours after application. The treatment mortality corrected for control mortality (M) is calculated using Abbot's Formula (Abbot, W. S. (1925): A method of Computing the Effectiveness of an Insecticide, Journal of Economic Entomology, 18, 265-267)

M=((mₜ−m_{c})/(100−m_{c}))×100

mₜ being the mortality of pests treated with the active material and m_{c} being the mortality of pests treated with the control material.

The pesticide preferably contains a higher concentration of soap than fragrance. In preferred embodiments the soap and fragrance are in weight ratio of at least about 1:1, preferably at least about 5:1, more preferably from about 6:1 to about 40:1, and especially from about 8:1 to about 20:1. The soap and fragrance concentrations are preferably such as to avoid the formation of an emulsion, ie, one separable by centrifugation or ultracentrifugation. The soap is preferably selected from alkali metal salts of carboxylic acids containing at least 10 carbon atoms and its concentration is preferably less than about 5%, preferably less than about 3% by weight of composition, and preferably at least about 0.2%, more preferably at least about 1% by weight of composition.

Preferably, the fragrance herein comprises one or more terpenoid natural oils (and their synthetic equivalents), the levels of terpenoid fragrance components by weight of the fragrance being preferably at least about 20%, more preferably at least about 40% and especially at least about 60%. Suitable fragrances herein include natural oils such as D-limonene, linalool, eugenol, geraniol, anethole, carvacrol, citronellal, D-pulegone, alpha-terpineol, thymol, borneol and mixtures thereof. Preferred natural oils for use herein include D-limonene, linalool, mixtures of D-limonene and linalool, eugenol, geraniol and mixtures of eugenol and geraniol. Especially preferred is the use of geraniol. The fragrance concentration is preferably less than about 1%, more preferably less than about 0.6% by weight of composition and is preferably at least about 0.02%, more preferably at least about 0.1% by weight of composition. The pesticide can further comprise a conventional insecticidal synergist such as piperonyl butoxide.

In a preferred embodiment, the fragrance additionally comprises one or more insect-repellant fragrance components. Preferably the fragrance comprises at least about 1%, more preferably at least about 3% and especially at least about 5% of one or more insect-repellant components. The soap/fragrance combination is effective in maintaining the application area free of insects and provides a pleasant odor for a period of at least about 7 days preferably at least about 10 days and more preferably at least about 14 days.

Thus, according to another aspect of the invention, there is provided a liquid pesticide comprising an insecticidal soap and a fragrance, and wherein the fragrance comprises one or more terpenoid natural oils as insecticidal synergist and optionally one or more insect-repellant fragrance components.

In preferred embodiments the pesticide further comprises an evaporation retardant. Thus, according to another aspect of the invention, there is provided a liquid pesticide comprising an insecticidal soap, a fragrance and an evaporation retardant. By “evaporation retardant” is meant a substance that has the capacity to reduce the rate of drying of the pesticide on a plant substrate and/or to enhance liquid retention by the pesticide on the substrate. Although the active components can be formulated in any liquid carrier including organic and inorganic solvents, water is preferred in the present application.

The pesticide preferably contains at least equal or higher concentration of soap to evaporation retardant. In preferred embodiments the soap and the evaporation retardant are in weight ratio of at least about 1:1, preferably at least about 5:1. The soap is preferably selected from alkali metal salts of carboxylic acids containing at least 10 carbon atoms and its concentration is preferably less than about 5%, more preferably less than about 3% and about 2% by weight of the composition.

Suitable evaporation retardants include humectants, thickeners and mixtures thereof. Humectants are capable of absorbing moisture at certain humidity levels and of reducing the rate of moisture evaporation from the liquid pesticide on the treated substrate. Suitable humectants herein include glycols and other polyhydroxy alcohols such as glycerol and sorbitol; potassium polymetaphosphate, sodium chloride, triacetin and mixtures thereof.

Thickeners retard the evaporation by changing the viscosity of the liquid pesticide. Suitable thickeners herein include i) polymeric thickeners, such as polycarboxylate synthetic polymer preferably cross-linked and with a molecular weight at least about 500,000; ii) natural gums, such as xanthan gum, locust bean gum, guar gum, arabic gum, karaya gum, tragacanth gum, agar gum, algin gum, carrageenan gum, gellan gum and the like; iii) semi-synthetic thickeners such as the cellulosic type thickeners: hydroxymethyl and hydroxyethyl cellulose; iv) pectins; and iv) mixtures thereof. Highly preferred herein are thickeners which themselves have a humectant property. In a preferred embodiment xanthan gum is used as evaporation retardant.

The pesticides of the invention comprising an evaporation retardant also comprise a fragrance, preferably a fragrance as described herein above which synergistically re-enforces the insecticidal or other pesticidal activity of the soap. Suitable fragrances herein include natural oils such as D-limonene, linalool, eugenol, geraniol, anethole, carvacrol, citronellal, D-pulegone, alpha-terpineol, thymol, borneol and mixtures thereof.

In other embodiments the pesticide additionally comprises a particulate, physically-active insecticidal material. The soap acts to enhance the residuality of the particulate material to a target substrate, apparently by an adhesive mode of action.

Thus, according to another aspect of the invention, there is provided a liquid pesticide comprising a particulate, physically-active insecticidal material and a water-soluble surface-active adhesive for enhancing the residuality of the insecticidal material to a target substrate. Although the actives components can be formulated in any liquid carrier including organic and inorganic solvents, water is preferred in the present application. Accordingly, the pesticides herein based on particulate, physically-active insecticidal materials are preferably in the form of an aqueous dispersion of the particulate insecticide in an aqueous surfactant solution.

Mechanistically, it is believed that the particulate physically-active material becomes trapped by the bodies of the insect as they walk over it. The particulate solid damages the insects exoskeleton mainly by absorbing lipid molecules from their waxy epicuticle. Abrasion can also play an important role, puncturing the insect exoskeleton. The result is the loss of water from the insect's body leading to a desiccation of the insect resulting in death. Optimum efficacy is obtained when the particle size of the insecticide is below 50 μm, preferably below 30 μm. In general, the particulate, physically-active insecticidal material can be described as a lipid absorbent material having a high absorptivity surface area able to absorb up to four times its weight of liquid and a particle size below about 100 μm, preferably below about 50 μm and more preferably below about 30 μm.

The hardness of the particulate solid is an important factor determining the abrasive capacity of the particles. In preferred embodiments the particulate insecticidal material used herein is selected from natural and synthetic abrasives having a Mohs hardness of less than about 7, preferably less than about 5 and more preferably less than about 3. The particulate material for use herein is preferably selected from calcium carbonate, mica, talc, kaolin, bentonites, clays, attapulgite, pyrophyllite, wollastonite, silica, feldspar, sand, quartz, chalk, limestone, barytes, diatomaceous earth, synthetic inorganic or organic materials and mixtures thereof. In preferred embodiments diatomaceous earth is used as particulate insecticidal material. The spiny structure of this material ensure a good insecticidal performance.

The role of the surface-active adhesive material is believed to be three-fold. Firstly, it provides a good suspension of the particulate material in the liquid pesticide giving rise to a uniform delivery of the particulate material. Secondly, once the pesticide is delivered the surface-active adhesive material gives stickiness to the particulate material resulting in an increase in residuality. Thirdly, the surface-active adhesive material can act to soften the insect cuticle, thereby promoting the efficacy of the particulate insecticide. The surface-active adhesive is preferably selected from alkali metal salts of carboxylic acids containing at least 10 carbon atoms and its concentration is preferably less than about 5% and more preferably less than about 3% per weight of the pesticide composition.

Thus, according to another aspect of the invention, the liquid pesticide comprises a particulate abrasive having a Mohs hardness of less than about 7 and an insect cuticle softening agent. According to a further aspect of the invention, there is provided a liquid pesticide comprising from about 0.01 to about 2% by weight of diatomaceous earth and form about 0.2 to about 5% of alkali metal natural soaps.

The pesticide can also contain known perfume auxiliaries in order to make its use more pleasant from the user point of view.

The pesticides described herein are useful for controlling insects and related arthropods. Although the pesticide is useful for controlling insects on plants it can also be applied to any other insect infested hosts, such as pets or household surfaces. In preferred embodiments the pesticides are applied to garden, house and greenhouse plants and to vegetables and crops.

### DETAILED DESCRIPTION

The present invention envisages in one aspect pesticides comprising soap, fragrance and additionally other known components of pesticidal compositions. In another aspect, the present invention envisages pesticides comprising soap, fragrance and evaporation retardant. Liquid pesticides comprising a particulate, physically-active insecticidal material and a water-soluble surface-active adhesive are also envisaged according to a further aspect of the invention. Regarding the soap either one, or preferably, a mixture of fatty acid salts are normally employed. Preferred fatty acid salts are those having between eight and twenty carbon atoms in a straight chain structure with the alpha carbon comprising a monocarboxylic acid moiety esterified, preferably completely, with a monovalent metal such as sodium or potassium. Alkanol ammonia and ammonia can also be used as counter-ion for the salts. This group of fatty acid salts are known to have insecticidal activity, and have been used for many years in the control of pestiferous arthropods. Fatty acid soap materials are widely commercially available. They can be produced from coconut oils, comprising predominantly a mixture of laurate (C-12) and myristate (C-14). They are also derived from various plant and animal sources. The preferred fatty acids are those having eight to eighteen carbon atoms including caprylate, laurate, myristate, palmitate, oleate, linoleate and stearate. Most preferred are unsaturated, eighteen carbon atom salts such as alkali metal oleate and linoleate, and saturated eight to twelve carbon atom salts such as mixtures of alkali metal caprylate, pelargonate, caprate, undecylinate and laurate.

The active insecticidal components can be dispersed in any organic or inorganic solvent. Specially useful are alcohols such as methanol, ethanol, glycerol, isopropyl alcohol, mixtures thereof and mixtures of these alcohols with water. Aqueous solutions are highly preferred. The pH of the aqueous solutions should be such as to allow the soap to be in its salt form and to be solubilised. The pH of the aqueous solutions is preferably neutral or alkaline, i.e., from about 7 to about 11, more preferably from about 9 to about 10.

Useful fragrances for use herein include essential oils such as terpenoids. The name terpenoid is applied in a general sense to certain substances naturally occurring in plants, the great majority of which possess carbon skeletons which can be regarded as built up on a basis of the fusion of two or more isoprene units. The terpenoids are classified depending on the number of isoprene units in their carbon skeleton. Hemi-, mono-, sesqui-, di-, sester-, tri-, tetra- and poly-terpenoids have 1, 2, 3, 4, 5, 6, 8 and more than 8 isoprene units, respectively. Highly preferred for use herein are mono-terpenoids. They are found in the essential oils obtained from plants and trees. They show a wide diversity of oxygen function and include alcohols, aldehydes, ketones, oxides, etc. Among the mono-terpenoids suitable for use herein are D-limonene, linalool, geraniol, eugenol, anethole, carvacrol, citronellal, D-pulegone, alpha-terpineol, thymol and borneol. Terpene alcohols are especially preferred herein.

D-limonene is a water white to slightly yellow mono-cyclic terpene obtained as a byproduct of the manufacture of citrus molasses. It is obtained by steam distillation of citrus peels and pulp resulting from the production of citrus juices and is referred to as a “stripper oil,” which contains about 94-98% of the D-limonene.

Linalool occurs naturally in more than 200 oils from flowers, wood, leaves and herbs. It is found in oils of Ceylon's cinnamon, sassafras, orange flower, bergamot, Artemisia balchanorum, ylang ylang, rosewood and other oils. Linalool is commonly used as a flavouring agent and as a perfume. Linalool is also known as lily of the valley scent, coriandrol or 3,7-dimethyl-1,6-octadiene-3-ol. Linalool may be obtained directly by fractional distillation from vegetable products. It can also be obtained synthetically from other monocyclic terpenes. It can be distilled from commercial D-limonene, the commonly available form of which may comprise on the order of 95 percent pure D-limonene and approximately seven other component oils.

Other fragrances suitable for use herein include: amyl cinnamic aldehyde, amyl salicylate, anisic aldehyde, benzyl acetate, cinnamic alcohol, diethyl phthalate, dipropylene glycol, ionone, methyl anthranilate, methyl ionone, phenyl ethyl alcohol, terpinyl acetate, 4-tert butylcyclohexyl acetate and mixtures thereof.

Other fragrances with insect repellent properties suitable herein include methyl dihydro jasmonate, ajowan oil, basil oil, black pepper oil, cardamon oil, carrot seed oil, cedarwood oil, celery seed oil, cinnamic aldehyde, citronella oil, coniferyl aldehyde, croton elutria oil, eucalyptus citriodora oil, farlic iol, ginger oil, hexylcinnamic aldehyde, hyssop oil, laurel oil, lavender oil, L-carvone, marjoram oil, mint oil, neem oil, onion oil, pennyroyal oil, pepper oil, petitgrain oil, resemary oil, sage oil, spearment oil, tagetes oil, tansy oil, thyme oil, thymol, white pepper oil and mixtures thereof.

Fragrances suitable for use herein are described generally in Perfumes and Flavour Chemicals and Perfume and Flavour Materials of Natural Origin, both by Steffen Arctander.

The liquid pesticides described herein can be supplemented by other known components of liquid pesticides including sequestering and chelating agents, wetting agents, perfumes and fragrances, humectants and evaporation retardants, thickening agents, binders, insect repellents and attractants, insect growth regulators, chitin inhibitors, insect pathogens, plant self-defence induction agents, aphid alarm pheromones, antioxidants and preservatives.

Sequestering and chelating agents can be used to enhance the insecticidal and other pesticidal activity. They act to remove excess metal ions (e.g., calcium, copper, iron, manganese). Broadly, the nature of the sequestering agent used herein determines the optimal concentration of sequestering agent in a given preparation. Generally, the ratio of fatty acid salts to sequestering agent, on a weight basis, can vary between 1:0.05 to 1:5, but usually the agent is present in amounts no greater than the fatty acid salt content. Ratios outside these ranges may be used, but result generally in barely significant improvement at the lower amounts of sequestering agent or reagent waste at the higher amounts. Among the chelating agents which may be used are carboxylic acid chelating agents such as citrate, gluconate, and ascorbate, and alkylenepolyamine polyacetic acids such as nitrilotriacetic acid, N-2-hydroxyethylaminodiacetic acid, ethylenediamine tetraacetic acid (EDTA), diethylene triamine penta acetic acid, N-2-hydroxyethyl ethylenediamine triacetic acid, propylene-1, 2-diamine tetracetic acid, propylene-1, 3-diamine tetracetic acid, and the isomeric butylenediamine tetraacetic acids. Either an alkali metal salt or alkanolamine salt of these chelating agents may be used. Another class of sequestering agents useful in the compositions herein are water-soluble phosphate-containing sequestering agents such as tripolyphosphates and phosphate salts and aminoorganic phosphonic acid compounds (e.g., ethylene diamine tetra(methylene phosphonic) acid, diethylene triamine penta(methylene phosphonic) acid). Excellent results have been observed using NH4 H2 PO4. Another class of agents which are capable of sequestering metal ions and may be used in the compositions described herein are anionic surfactants such as alkali metal or ammonium salts of lauryl alkanolamide sulphosuccinate, alkylarylpolyether sulphates and sulfonates, coco-isethionate and lignosulphonates. Also useful are amphoteric surfactants such as the water soluble salts of coco-betaine, coco-amphocarboxlyglycinate, coco-sulphobetaine, and imidazoline. Non-ionic surfactants such as alkylphenolethoxylate, and compounds known to be useful to sequester hard metal ions such as alkylphenolethoxylated phosphate, citrate, or fatty acid esters. Of course, compatible mixtures and blends of any of the foregoing may also be used. In summary, there is a wide variety of commercially available sequestering agents which may be used to formulate compositions embodying the invention.

As well as reducing the rate of moisture evaporation from the liquid pesticide on the treated substrate, humectants can be used to retard drying of the liquid carrier. Useful humectants to use herein are glycols and other polyhydroxy alcohols such as glycerol and sorbitol; potassium polymetaphosphate, sodium chloride, triacetin and mixtures thereof.

An additional component of the formulation can be a binder, preferably LIGNOSITE. LIGNOSITE is a trademark of Georgia Pacific Corporation of Houston, Tex. LIGNOSITE is a hygroscopic adhesive binder consisting of a wheaten or potato dextrin, or a calcium or sodium or ammonia salt of lignosulfonic acid.

An additional component of the formulation can be a wetting agent as for instance TRITON 101. TRITON 101 is a trademark of the Rohm and Haas Corporation of Philadelphia, Pa. TRITON 101 is a preferred chemical wetting agent or surfactant, a non-ionic surfactant compatible with the humectant and other components of the solutions described herein. Other wetting agents include TRITON X100, NINOL II-CN, IGEPAL 60630, nonyl phenol ethoxylate 9-15 mole and silicone surfactants such as Silwets. Silwets are a trademark of the CKWitco Corporation of New York.

The compositions herein preferably also include a thickener. By thickener is meant a component which has thickening properties, that is a compound which produces compositions with a higher viscosity in the presence of the thickener than in the absence of the thickener. Suitable thickener agents for use herein are: i) polymeric thickeners, such as polycarboxylate synthetic polymer preferably cross-linked and with a molecular weight at least about 500,000; ii) natural gums, such as xanthan gum, locust bean gum, guar gum and the like; iii) semi-synthetic thickeners such as the cellulosic type thickeners: hydroxymethyl and hydroxyethyl cellulose and iv) mixtures thereof. A preferred thickener is Aqualon. Aqualon is trademark of Hercules, Inc. of Wilmington, Del. Aqualon is a cellulose ether adhesive thickener.

Insect attractant suitable for the present invention may be selected from insect-attractive pheromones and scents and from insect-attractive ingestable materials, such as carbohydrates, proteins, fats, oils, inorganic salts, artificial sweeteners, vitamins, natural and artificial flavours, and any other attractant that can be carried by an aqueous liquid carrier and made accessible to insects. For any given target insect, a combination of attractants may be as or even more effective than the attractants individually.

Useful insect growth regulators include but are not limited to methoprene, fenoxycarb, and pyriproxyfen. Chitin inhibitors include but are not limited to flufenoxuron (sold as “Motto” by the Shell Chemical Company of Houston, Tex.) and lufenuron (as sold by the Ciba-Geigy Corp. of Greensboro, N.C.).

Living insect pathogens, such as insect pathogenic viruses, bacteria, fungi, or nematodes, also may be delivered to a target insect by inclusion in the liquid pesticide. Examples include strains of Baculovirus, other useful bacteria such as strains of Bacillus thuringiensis and Bacillus sphaericus, strains of the fungus Verticillium, and naturally occurring entomogenous nematodes, such as those sold under the mark “Biosafe” by Biosys, Inc. of Palo Alto, Calif. Alternatively, insect controlling materials may be derived or recovered from insect pathogens. Examples of such materials include the exotoxin crystals isolatable from B. thuringiensis; certain processed material from Xenorhabis spp. nematodes, and certain bacterial fermentation products such as avermectins and the product sold under the mark “DiBeta” by Abbott Laboratories of North Chicago, Ill. DiBeta, also known as “beta-exotoxin,” is reported to be a biological insecticide derived from the bacterial fermentation of a strain of B. thuringiensis. All types of insect control active ingredients may be used individually or in combination with other, compatible insect control active ingredients.

Suitable alarm pheromones to include in pesticides can be E-β-farnesene and α-pinene.

Plant defence mechanism can be induced by adding to the pesticide a defence protein inducing agent. Inducing agent suitable herein are jasmonic acid, lower alkyl esters of jasmonic acid or jasmonic acid-like derivatives compounds.

The compositions herein preferably include a particulate, physically active insecticidal material. Suitable particulate insecticidal materials include calcium carbonate, mica, talc, kaolin, bentonites, clays, attapulgite, pyrophyllite, wollastonite, silica, feldspar, sand, quartz, chalk, limestone, barytes, diatomaceous earth, synthetic inorganic or organic materials and mixtures thereof. In a preferred embodiment diatomaceous earth is used. Diatomaceous earth is a natural occurring siliceous sedimentary mineral compound from microscopic skeletal remains of unicellular algae-like plants called diatoms. These plants have been part of the earth's ecology since prehistoric times. As living plants, diatoms weave microscopic shells from the silica they extract from the water. Then as they die, deposits are formed and eventually fossilised in what are now dried lake and ocean beds. The material is then mined, ground and screened to various grades. Silicon dioxide is the major component in diatomaceous earth, generally representing about 80% by weight of the composition. Other components include calcium oxide, aluminium oxide, iron oxide and other oxides and oligo-elements. In turn of physical properties, diatomaceous earth has high porosity (up to eighty-five percent of the volume of diatomaceous earth is made up of tiny interconnected pores and voids), high absorptivity (diatomaceous earth can generally absorb up to four times its own weight in liquid and still exhibit the properties of dry powder), high surface area (approximately 30 m²/g) and is very irregular in shape, having generally spiny structures and pitted surface area. Diatomaceous earth is commercially available under trademarks such as Celite™, Di-atomate™ and Prosper®.

Regarding the water-soluble surface-active adhesive, a fatty acid salt or a mixture of fatty acid salts are normally employed, preferred fatty acid salts being described in detail above. Unexpectedly, the fatty acid salts are found to be effective in combination with the particulate physical insecticide in liquid pesticidal compositions to provide enhanced insecticidal efficacy and residuality, as well as a more homogeneous distribution of the particulate insecticide over the surface of the target substrate. This is especially surprising, given that the particulate physical insecticides are generally believed to be ineffective in liquid formulations.

The liquid pesticide is efficient against insects commonly found on ornamental plants, vegetable crops and fruit trees. Insects found in a garden environment includes, aphids, armyworms, caterpillars, chinch bugs, flea beetles, fleahoppers, flies, fruit flies, japanese beetles, leafhoppers, leafminers, leafrollers, loopers, lygus bugs, mealy bugs, mites, plant bug, thrips, white flies, 12-spotted cucumber beetles, beet webworms, blister beetles, cabbage looper, cabbage worms, celery leaftiers, potato beetle, corn earworm, cucumber beetles, diamondback moth larvae, european corn borer, harlequin bugs, mexican bean beetles, oblique-banded leafrollers, plant stink bugs, squash vine borers, thrips, vegetable weevils, etc.

### EXAMPLES

The following are examples of liquid pesticides according to the invention. The compositions are given in percentage per weight.

| | I | II | III | IV | V |
|---|---|---|---|---|---|
| Potassium | 2 | 2 | 2 | 2 | 2 |
| oleate | | | | | |
| Geraniol | 0.3 | 0.3 | | 0.15 | 0.2 |
| Eugenol | | | 0.3 | 0.15 | 0.1 |
| Xanthan | | 0.01 | 0.05 | 0.1 | 0.1 |
| gum | | | | | |
| Diat. earth | | | 0.1 | 0.1 | |
| Antioxidant | | 0.01 | 0.02 | 0.01 | 0.02 |
| Preservative | | 0.05 | 0.02 | 0.01 | 0.02 |
| Perfume | | 0.01 | 0.01 | 0.01 | 0.01 |
| piperonyl | | 0.01 | 0.01 | | |
| butoxide | | | | | |
| Distilled | To 100 | To 100 | To 100 | To 100 | To 100 |
| water | | | | | |
| | VI | VII | VIII | IX | X |
| Potassium | 2 | 2 | 2 | 2 | 2 |
| oleate | | | | | |
| Diat. earth | 2 | 0.9 | 1.5 | 1.2 | 1 |
| Xanthan | | 0.01 | 0.05 | 0.1 | 0.1 |
| gum | | | | | |
| Geraniol | | 0.3 | | | 0.2 |
| Eugenol | | | 0.3 | | 0.1 |
| Antioxidant | | 0.01 | 0.02 | 0.01 | 0.02 |
| Preservative | | 0.05 | 0.02 | 0.01 | 0.02 |
| Perfume | | 0.01 | 0.01 | 0.01 | 0.01 |
| piperonyl | | 0.01 | 0.01 | | |
| butoxide | | | | | |
| Distilled | To 100 | To 100 | To 100 | To 100 | To 100 |
| water | | | | | |
| | XI | XII | XIII | XIV | XV |
| Potassium | 2 | 2 | 2 | 2 | 2 |
| oleate | | | | | |
| Geraniol | 0.15 | 0.1 | | 0.15 | 0.15 |
| Eugenol | | 0.1 | 0.15 | | 0.05 |
| Xanthan | | 0.01 | 0.05 | 0.1 | 0.1 |
| gum | | | | | |
| Diat. earth | | 0.7 | 0.5 | | |
| Antioxidant | | 0.01 | 0.02 | 0.01 | 0.02 |
| Preservative | 0.9 | 0.5 | 0.7 | 1.0 | 0.5 |
| Perfume | 0.05 | 0.03 | 0.1 | 0.03 | 0.15 |
| piperonyl | | | 0.01 | | |
| butoxide | | | | | |
| Distilled | To 100 | To 100 | To 100 | To 100 | To 100 |
| water | | | | | |

In the above, ‘perfume’ is a combination of natural and synthetic fragrance components having insect repellant properties and incorporating, inter alia, methyl dihydro jasmonate, orange oil, petitgrain, bergamot juice phase oil, grapefruit phase oil, mandarin oil, and dihydro myrcenol. ‘Preservative’ is a 50/50 mix of benzyl alcohol and phenylethyl alcohol. The compositions of Examples I to XV have excellent insecticidal efficacy against a range of foliar pests as well as an excellent safety profile for both humans and plants.

What is claimed:

1. A liquid pesticide comprising:
an insecticidal soap solution, said insecticidal soap solution having a fragrance solubilized therein; and,
wherein said insecticidal soap solution and said fragrance are present in said liquid pesticide in a weight ratio ranging from about 6:1 to about 40:1.

2. The liquid pesticide of claim 1 wherein said insecticidal soap solution is selected from the group consisting of carboxylic acids containing at least about 10 carbon atoms, alkali metal salts thereof, and mixtures thereof.

3. The liquid pesticide of claim 2 wherein said insecticidal soap solution concentration ranges from at least about 0.2 percent by weight to less than about 5 percent by weight of composition.

4. The liquid pesticide of claim 1 wherein said fragrance comprises at least one oil selected from the group consisting of D-limonene, linalool, eugenol, geraniol, anethole, carvacrol, citronellal, D-pulegone, alpha-terpineol, thymol, borneol, and mixtures thereof.

5. The liquid pesticide of claim 4 wherein said fragrance has a concentration ranging from at least about 0.1 percent by weight of composition to less than about 1 percent by weight of composition.

6. The liquid pesticide of claim 1 wherein said liquid pesticide is an isotropic and thermodynamically stable micellar solution.

7. The liquid pesticide of claim 1 wherein said fragrance comprises at least about 1 percent of insect-repellant fragrance components.

8. The liquid pesticide of claim 7 wherein said fragrance comprises at least one perfume auxiliary.

9. The liquid pesticide of claim 1 wherein said liquid pesticide is provided as a liquid pesticidal concentrate.

10. A liquid pesticide comprising:
(a) an insecticidal soap;
(b) an evaporation retardant, said evaporation retardant being selected from the group consisting of humectants, thickeners, and mixtures thereof; and,
(c) a fragrance, said fragrance comprising at least one terpinoid and a natural oil selected from the group consisting of D-limonene, linalool, eugenol, geraniol, anethole, carvacrol, citronellal, D-pulegone, alpha-terpineol, thymol, borneol and mixtures thereof; and,
wherein said insecticidal soap and said evaporation retardant are provided in a weight ratio of at least about 1:1.

11. The liquid pesticide of claim 10, wherein said insecticidal soap is selected from the group consisting of carboxylic acids containing at least about 10 carbon atoms, alkali metal salts thereof, and mixtures thereof.

12. The liquid pesticide of claim 11 wherein said insecticidal soap has a concentration of less than about 3 percent by weight of composition.

13. The liquid pesticide of claim 10 wherein said thickener is selected from the group consisting of polymeric thickeners, natural gums, semi-synthetic thickeners, pectins, and mixtures thereof.

14. The liquid pesticide of claim 10 wherein said evaporation retardant is xanthan gum.

15. The liquid pesticide of claim 10 further comprising a physically active particulate insecticidal material.

16. The liquid pesticide of claim 10 wherein said liquid pesticide is provided as a liquid pesticidal concentrate.

17. A liquid pesticide comprising a physically-active, particulate insecticidal material and a water-soluble surface-active adhesive, wherein said adhesive enhances residuality of said insecticidal material to a target substrate.

18. The liquid pesticide of claim 17 further comprising from about 0.01 percent by weight to about 2 percent by weight of diatomaceous earth and from about 0.2 percent by weight to about 5 percent of alkali metal natural soap or mixtures of soaps.

19. The liquid pesticide of claim 17 further comprising a fragrance, wherein said fragrance comprises at least one terpenoid natural oil.

20. The liquid pesticide of claim 19 wherein said liquid pesticide further comprises at least one insect-repellant fragrance component.
